(19) United States
(12) Patent Application Publication
(10) Pub. No.: US 2006/0048784 A1
(43) Pub. Date: Mar. 9, 2006

(54) Two layered condom

(76) Inventor: John Andrew Turner, Friendswood, TX (US)

Correspondence Address: John Andrew Turner, 714 Stonecreek Circle, Friendswood, TX, 77546, US

(21) Appl. No.: 10/935,523

(22) Filed: Sep. 7, 2004

(51) Int. Cl. A61F 6/04

(52) U.S. Cl. 128/844

(57) ABSTRACT

An improved two-layered condom device which offers improved sensation to the male wearer compared to a conventional condom, which should result in increased compliance with usage. The device provides a dual barrier, with lubricant between an inner condom and an outer condom which may contain a chemical or chemicals to neutralize bacteria, viruses, and sperm, thereby making it superior to a conventional single-layered condom in preventing unwanted pregnancy and the spread of sexually transmitted diseases.

### FIELD OF THE INVENTION

The present invention relates to condoms and, more particularly, to condoms which provide enhanced tactile stimulus transmission to the wearer and increased protection from unwanted pregnancy and the spread of sexually transmitted diseases.

### BACKGROUND OF THE INVENTION

Condoms have been around for centuries, and are well established as being effective as contraceptives when used properly. Short of abstinence, a condom, if used correctly, provides the most effective defense against sexually transmitted diseases (STDs), which distinguishes it from other methods of birth control. Preventing transmission of STDs is becoming increasingly important given their prevalence, morbidity, and mortality. These STDs include chlamydia, gonorrhea, nongonococcal urethritis, trichomoniasis, herpesviruses, human papillomaviruses (which cause genital warts, and some types cause cervical cancer in women), syphilis, Hemophilis ducreyi infection, hepatitis B and C, and human immmunodeficiency virus (HIV, the etiological agent in acquired immunodeficiency disease, or AIDS), as well as STDs which have yet to be discovered. Chemical compounds which have bactericidal, virucidal, and spermicidal properties, such as nonoxynol-9, have been packaged in the form of a lubricant with condoms in hopes of augmenting the protection against both pregnancy and STD transmission, but nonoxynol-9 has recently been determined to not offer protection against STDs, so other chemicals are being sought to offer additional protection.

Given the facts that correctly used condoms are effective at preventing pregnancy and are the most effective means short of abstinence for preventing transmission of STDs, more widespread and consistent use of them by those who are promiscuous or do not desire pregnancy could potentially have a substantial positive impact on public health. The reason condom use is not more widespread is that the wall of a condom, which is a barrier-between sexual partners possessing the protective qualities listed above also becomes a barrier which reduces the tactile sensitivity between the two partners, thereby reducing the pleasure of coitus, especially for the male condom wearer. Coitus is a powerful, spontaneous, and impulsive act in humans, and because rational thought tends to be diminished during this time, irrational decisions—and acts—often follow. Since use of a conventional condom diminishes sensation, and thereby pleasure, of the wearer, its use is too often avoided. An unused condom is an ineffective condom, so it follows that a condom with properties allowing for enhanced tactile stimulus transmission to the male wearer would be more likely to be used, and used consistently. This increased usage would likely result in less disease spread and unwanted pregnancy, and could benefit society on a large scale from a public health perspective. Preventing even one transmission of HIV would make this invention worthwhile.

Other solutions in existence include:

1. Conventional condom.

2. Diaphragm, cervical cap.

3. Oral contraception.

4. Surgical sterilization.

5. Intrauterine device.

Shortcomings of other solutions include:

1. Decreased tactile sensation of the wearer often leads to decreased usage.

2. Suboptimal birth control method with inadequate protection against transmission of STDs.

3. No protection against STDs and must be taken daily (only available for females, at this time) without missing a dose to maintain effectiveness as a method of birth control.

4. No protection against STDs and imparts the inherent risks of surgery to the recipient; more risky for a female than a male.

5. No protection against STDs but has an increased risk of pelvic infections and possibly of future infertility.

It is therefore an object of the invention to provide an improved barrier method of birth control with improved protection against the spread of STDs which also provides for enhanced tactile stimulus transmission to the male wearer over conventional condoms.

It is another object of the invention to provide a device comprising two barrier layers which contain a lubricant which may carry a chemical or chemicals that serve to neutralize STDs and sperm.

It is another object of the invention to provide projections incorporated within the inward facing side of the outer condom to accentuate tactile stimulus transmission to the male condom wearer.

### SUMMARY OF THE INVENTION

In accordance with the present invention, there is provided an improved two layered condom device which employs the use of projections on the inward facing wall of an outer condom to offer enhanced tactile stimulus transmission, or sensation, to a male wearer when compared to current conventional condoms. Because this device has two barriers, which contain a lubricant between an inner condom and an outer condom which may carry chemical substances which serve to neutralize bacteria, viruses, and sperm, it follows that it would be superior to a conventional condom in preventing sexually transmitted disease (STD) transmission and unwanted pregnancy. These qualities of affording an improved sensory experience to the male wearer as well as improved protection against unwanted pregnancy and the spread of STDs create a higher likelihood that this device will be used consistently, with all of the public health benefits inherent to consistent use of such a barrier device.

### BRIEF DESCRIPTION OF THE DRAWINGS

A complete understanding of the present invention may be obtained by reference to the accompanying drawings, when considered in conjunction with the subsequent, detailed description, in which:

FIG. 1 is a perspective view of an unrolled inner condom;

FIG. 2 is a perspective view of an unrolled outer condom;

FIG. 3 is a cross sectional view of a wall of an outer condom;

FIG. 4 is a perspective view of an outer condom positioned over an inner condom;

FIG. 5 is a perspective view of an alternative embodiment showing an inner condom and an outer condom bonded at a mutual condom base; and

FIG. 6 is a cross sectional view of a projection arising from the inward facing wall of an outer condom creating a deflection through the wall of an inner condom.

For purposes of clarity and brevity, like elements and components will bear the same designations and numbering throughout the FIGURES.

### DESCRIPTION OF THE PREFERRED EMBODIMENT

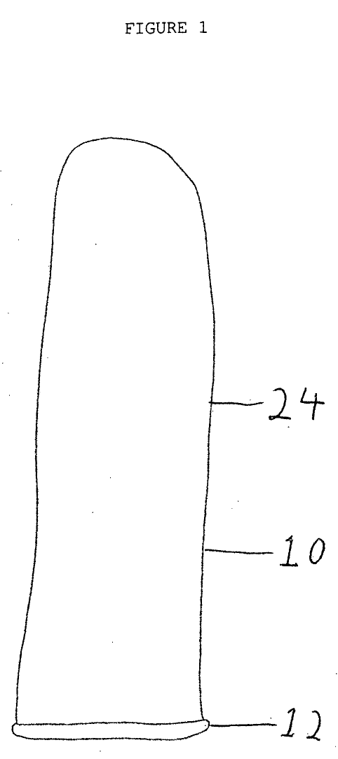

FIG. 1 shows a perspective view of an unrolled inner condom 10, which is in essence a conventional condom with a thin wall 24 made of latex, polyurethane, or other flexible material impermeable to fluid as is currently used in conventional condoms. The condom base 12 of said inner condom 10 is a thickened circumferential area at the proximal end of the unrolled condom which helps the condom retain its position on the penis of the wearer (not shown) during use of a device 22. It is rolled and packaged as is currently the practice for high quality condoms such as Trojan (R) brand condoms and others. No drawings are included to show a condom in its packaged state, as the only difference between the inner condom 10 and a conventional condom is that the package will be labeled so that the wearer will be aware of the order of placement of this condom.

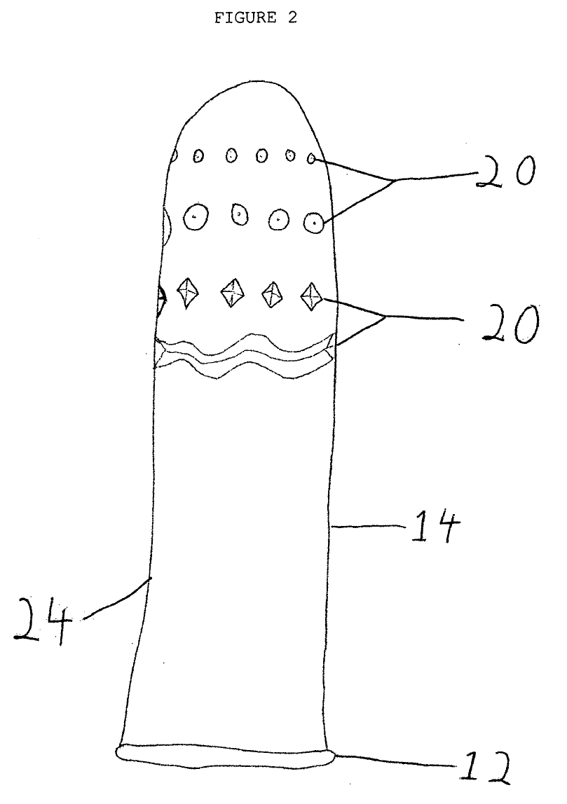

FIG. 2 is a perspective view of an outer condom 14. Although each projection 20, which is a convexity of any conceivable or practical size or shape, faces the inward facing inner lumen of the wall 24 of an outer condom 14, each projection 20 is visible from an external view, and is represented in this drawing. Like said inner condom 10, said outer condom 14 also has a condom base 12, which serves the same purpose of helping to retain the device 22 in position on the penis of the wearer. Said outer condom 14 is also made of flexible material impermeable to fluid, and, except for having projections in its wall 24 facing its inner lumen, is similar to conventional condoms. Said outer condom 14 is also rolled and packaged as conventional condoms, and the package is labeled to inform the wearer that this outer condom 14 is placed over said inner condom 10 after said inner condom 10 has been applied to the erect penis of the wearer.

Lubricant 18 in a sufficient quantity is placed during packaging of the outer condom 14 such that it will be in the inner lumen, or inward facing side, when a device 22 is installed, of said outer condom 14, and when said outer condom 14 is unrolled over said inner condom 10, a potential space 16 will be created which will contain said lubricant 18. Said lubricant 18 may also contain a chemical or chemicals which function to neutralize sperm and the causative agents of sexually transmitted diseases (STDs). Nonoxynol-9 is a chemical which gained Food and Drug Administration approval for use many years ago as a spermicide, and also has significant neutralizing activity against the etiological agents of many STDs in a laboratory setting, and was therefore widely thought to help to control the spread of STDs when used topically or in conjunction with condoms or diaphragms. But based on an extensive review of the literature, scientific opinion has reversed recently such that nonoxynol-9 is felt to offer no additional protection against STD transmission when used with conventional barrier methods of birth control. Because frequent exposure of this chemical to the vaginal mucosa has been associated with erosion and breakdown of this tissue, and said erosion may actually facilitate STD transmission, the Centers for Disease Control and Prevention has since discouraged use of nonoxynol-9 with barrier methods, although it is still in common use for its spermicidal properties.

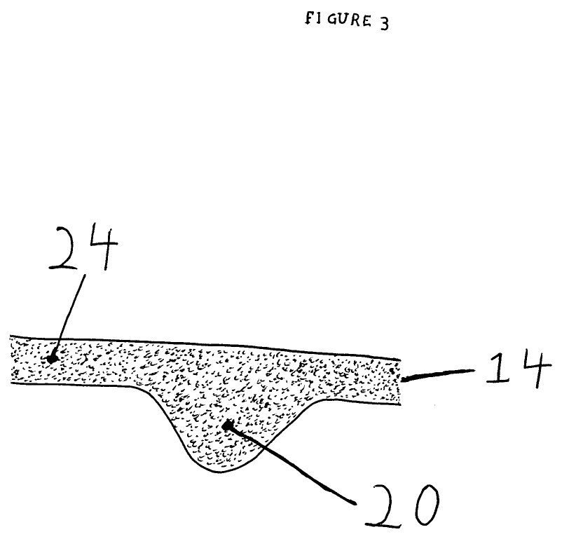

FIG. 3 is a cross sectional view of a wall 24 of the outer condom 14. Demonstrated in this figure is a projection 20, which is molded into the inward facing wall 24 of an outer condom 14 during the manufacturing process, using the same methods of manufacturing as is commonly used in making ribbed or textured conventional condoms.

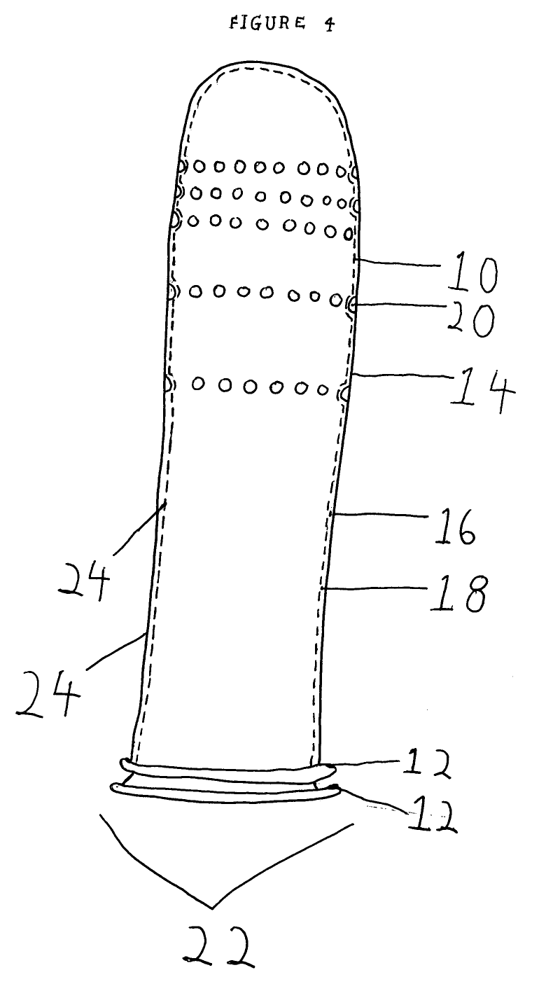

FIG. 4 is a perspective view of an outer condom 14 positioned over an inner condom 10, as it is to be positioned during use of a device 22. Detailed in this figure is a device 22 in its least expensive to manufacture embodiment, as it involves two separate condoms. An inner condom 10 is unrolled first onto an erect penis of the wearer. Then an outer condom 14 is unrolled onto said inner condom 10, with a lubricant 18 positioned in the inner lumen of said outer condom 14 during the manufacturing process of said outer condom 14 such that it occupies a potential space 16 created between said inner condom 10 and said outer condom 14. Said outer condom 14 is a slightly larger caliber, looser fitting condom than said inner condom 10 such that movement of said outer condom 14 over said inner condom 10 is not restricted. Shown are the inward facing projections of said outer condom 14, the potential space 16 which contains a lubricant 18, the wall 24 of said inner condom 10 and of said outer condom 14. Said inner condom 10 and said outer condom 14 each has a corresponding condom base 12 in this embodiment of a device 22.

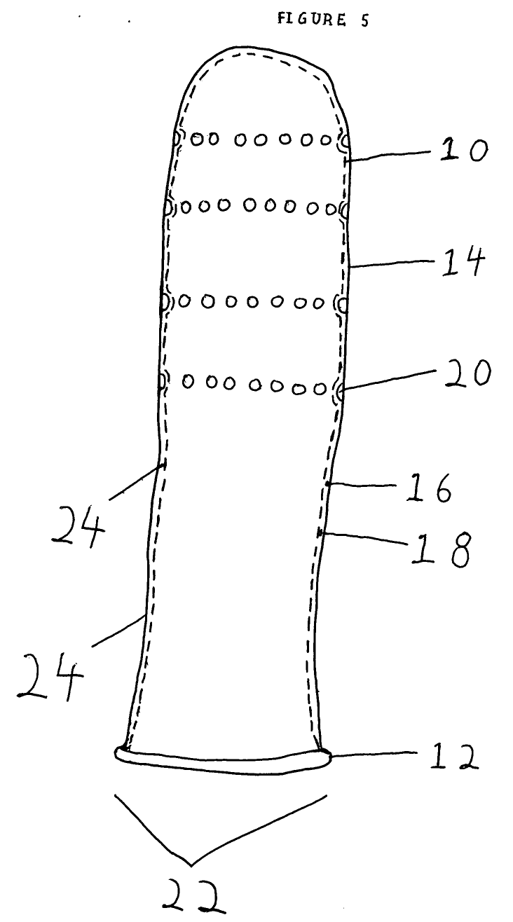

FIG. 5 is a perspective view of an alternative embodiment showing an inner condom 10 and an outer condom 14 bonded at a mutual condom base 12. In this alternative embodiment, a condom base 12 of said inner condom 10 and that of said outer condom 14 are joined while being manufactured such that said potential space 16 between said inner condom 10 and said outer condom 14 is sealed, and lubricant 18 is contained within said potential space 16. Although this embodiment is more expensive to manufacture, it can be rolled and packaged as a single unit so that a wearer will have no confusion as to the order of application for said inner condom 10 and said outer condom 14. Also, only having a single condom base 12, when compared to having two condom bases as the original embodiment has, may be more aesthetically pleasing for either the wearer of the device 22 or his sexual partner.

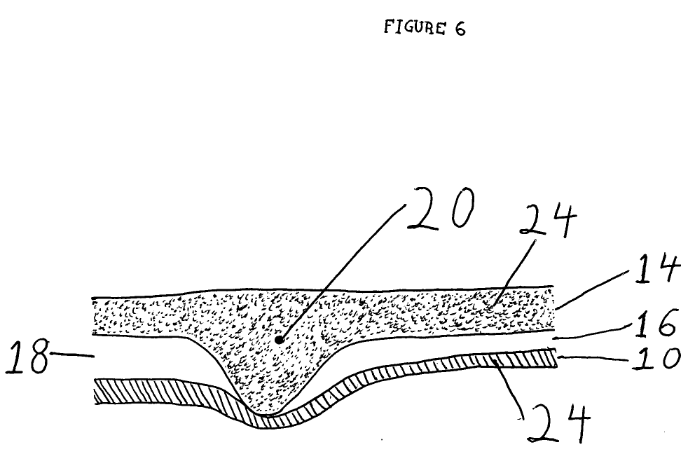

FIG. 6 is a cross sectional view of a projection 20 arising from the inward facing wall 24 of an outer condom 14 creating a deflection through the wall 24 of an inner condom 10. In this figure is shown a projection 20 on the inward facing wall 24 of an outer condom 14 deflecting a wall 24 of an inner condom 10 during use of a device 22. This deflection by a projection 20 transmits a tactile stimulus to the penis of the wearer, and is assisted by a lubricant 18 contained in a potential space 16 created between said inner condom 10 and said outer condom 14. Since a projection 20 does not exist alone, but rather in a plurality, multiple tactile stimuli are transmitted to the wearer of a device 22 during use.

Use of a device 22 is relatively straightforward. For application of the original embodiment of said device 22 FIG. 4), the wearer opens the clearly labeled package containing an inner condom 10, and unrolls it distally to proximally onto his erect penis in the usual fashion such that the condom base 12 is located circumferentially at the proximal part of his penis. Then the clearly labeled package containing an outer condom 14 is opened, and it is unrolled similarly over said inner condom 10, such that the base of said outer condom 14 is near said condom base 12 of said inner condom 10. A lubricant 18 is packaged in the inward facing lumen of said outer condom 14 such that when said outer condom 14 is installed, it occupies a potential space 16 created between said inner condom 10 and said outer condom 14. For application of an alternative embodiment of said device 22 (FIG. 5), the wearer opens a single package and unrolls said device 22 onto his erect penis using the same technique described herein above. In this embodiment, a common condom base 12 connects an inner condom 10 to an outer condom 14.

When a wearer of a device 22 engages in coitus, lubricant 18 contained in said potential space 16 provides for a lower coefficient of friction between the outward facing wall 24 of said inner condom 10 and the inward facing wall 24 of said outer condom 14 when compared with the coefficients of friction at both the interface of the penis of the wearer and the inward facing wall 24 of said inner condom 10, and at the interface of the outward facing wall 24 of said outer condom 14 and the sexual partner being engaged (FIG. 6). This, assisted by the relatively loose fit of said outer condom 14 over said inner condom 10, allows each projection 20, which is pushed circumferentially inward toward the wearer of said device 22 by external pressure from said sexual partner, to also be dragged across the relatively slick outward facing wall 24 of said inner condom 10. This dragging of each said projection 20 across a thin inner condom 10 causes a corresponding moving deflection onto the penis of the wearer, and thereby transmits a tactile stimulus to him in keeping with the rhythm of coitus. When coitus has concluded but before the penis of the wearer becomes non-erect, he must disengage from his sexual partner and carefully remove and discard said device 22.

It is counterintuitive that a dual barrier condom would have improved tactile stimulus transmission to a wearer when compared to a conventional single barrier condom, but designing each projection 20 to be as aggressively shaped as is necessary or desired, potentially much more aggressive or peaked than a conventional textured condom would have a need to be, is partly how this is achieved. That, combined with said lubricant 18, facilitates tactile stimulus transmission to the wearer by causing the appropriate surfaces to be more slick, as described herein above. Because this inventive device 22 comprises two distinct barriers, should there be a break in the wall 24 of either an inner condom 10 or of an outer condom 14, the remaining intact layer, combined with the presence of said lubricant 18 contained between said inner condom 10 and said outer condom 14, which may carry a chemical or chemicals which neutralize sperm and the causative agents of STDs, will continue to provide protection against unwanted pregnancy and STD transmission. Since breakage of a wall 24 of either said inner condom 10 or said outer condom 14 would be infrequent, and because very little, if any, said lubricant 18 would come in contact with either the wearer of a device 22 or his partner while using either the original or the alternative embodiment of said device 22, the potential exists for using chemicals or concentrations of chemicals which might not be considered for use with a conventional condom, and these chemicals could potentially possess extraordinary protection properties. All of these factors—the dual barrier with an inner condom 10 and an outer condom 14, said lubricant 18 between said inner condom 10 and said outer condom 14, and the enhanced stimulus transmission to the wearer of a device 22—make said device 22 more likely to be worn consistently and to be more effective protection against unwanted pregnancy and the spread of STDs when compared to a conventional condom.

Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention.

Having thus described the invention, what is desired to be protected by Letters Patent is presented in the subsequently appended claims.

What is claimed is:

1. A two layered condom for enhancing tactile stimulus transmission to the wearer while improving protection from unwanted pregnancy and the spread of sexually transmitted diseases comprising:
means for providing an inner barrier through which tactile stimuli created by the deflection of a plurality of projections in an outer condom are transmitted to a wearer during use of a device;
means for providing a second, outer barrier to afford an extra measure of protection from the spread of sexually transmitted diseases and unwanted pregnancy while containing projections on its inner surface, which deflect through the wall of an inner condom during use of a device to create tactile stimuli for the wearer;
means for decreasing the coefficient of friction between an inner condom and an outer condom in such a way that it enhances deflection of projections through the wall of an inner condom and thereby enhances tactile stimulus transmission to the wearer while it also provides the potential for acting as a vehicle for a chemical or chemicals which function to neutralize sperm and the causative agents of sexually transmitted diseases; and
means for creating a tactile stimulus to the wearer of a device by deflecting the wall of an inner condom during use, integrally molded to said means for providing a second, outer barrier to afford an extra measure of protection from the spread of sexually transmitted diseases and unwanted pregnancy while containing projections on its inner surface, which deflect through the wall of an inner condom during use of a device to create tactile stimuli for the wearer.

2. The two layered condom in accordance with claim 1, wherein said means for providing an inner barrier through which tactile stimuli created by the deflection of a plurality of projections in an outer condom are transmitted to a wearer during use of a device comprises a condom barrier made of a smooth, flexible material impermeable to fluid, referred to as an inner condom.

3. The two layered condom in accordance with claim 1, wherein said means for providing a second, outer barrier to afford an extra measure of protection from the spread of sexually transmitted diseases and unwanted pregnancy while containing projections on its inner surface, which deflect through the wall of an inner condom during use of a device to creaté tactile stimuli for the wearer comprises a condom barrier made of a flexible material impermeable to fluid, referred to as an outer condom.

4. The two layered condom in accordance with claim 1, wherein said means for decreasing the coefficient of friction between an inner condom and an outer condom in such a way that it enhances deflection of projections through the wall of an inner condom and thereby enhances tactile stimulus transmission to the wearer while it also provides the potential for acting as a vehicle for a chemical or chemicals which function to neutralize sperm and the causative agents of sexually transmitted diseases comprises a liquid or viscous gel, referred to as lubricant.

5. The two layered condom in accordance with claim 1, wherein said means for creating a tactile stimulus to the wearer of a device by deflecting the wall of an inner condom during use comprises a convexity of any shape arising from the inner surface of an outer condom, referred to as a projection.

6. A two layered condom for enhancing tactile stimulus transmission to the wearer while improving protection from unwanted pregnancy and the spread of sexually transmitted diseases comprising:
a condom barrier made of a smooth, flexible material impermeable to fluid, referred to as an inner condom, for providing an inner barrier through which tactile stimuli created by the deflection of a plurality of projections in an outer condom are transmitted to a wearer during use of a device;
a condom barrier made of a flexible material impermeable to fluid, referred to as an outer condom, for providing a second, outer barrier to afford an extra measure of protection from the spread of sexually transmitted diseases and unwanted pregnancy while containing projections on its inner surface, which deflect through the wall of an inner condom during use of a device to create tactile stimuli for the wearer;
a liquid or viscous gel, referred to as lubricant, for decreasing the coefficient of friction between an inner condom and an outer condom in such a way that it enhances deflection of projections through the wall of an inner condom and thereby enhances tactile stimulus transmission to the wearer while it also provides the potential for acting as a vehicle for a chemical or chemicals which function to neutralize sperm and the causative agents of sexually transmitted diseases; and
a convexity of any shape arising from the inner surface of an outer condom, referred to as a projection, for creating a tactile stimulus to the wearer of a device by deflecting the wall of an inner condom during use, integrally molded to said outer condom.

7. The two layered condom as recited in claim 6, further comprising:
a condom base, for retaining the position onto the base of the penis of a wearer during use of a device on either an inner condom or an outer condom individually, or alternatively can potentially serve as a common connection at the bases of both an inner condom and an outer condom, circumferentially molded to said inner condom, and circumferentially molded to said outer condom.

8. The two layered condom as recited in claim 6, further comprising:
a potential space, for containing a lubricant, adjacently positioned to said inner condom, adjacently positioned to said outer condom, and fluidly retained to said lubricant.

9. The two layered condom as recited in claim 7, further comprising:
a potential space, for containing a lubricant, adjacently positioned to said inner condom, adjacently positioned to said outer condom, and fluidly retained to said lubricant.

10. The two layered condom as recited in claim 7, wherein said condom base is thickened area at the proximal end of a condom, referred to as a.

11. The two layered condom as recited in claim 8, wherein said potential space is space between an inner condom and an outer condom, referred to as a.

12. The two layered condom as recited in claim 9, wherein said condom base is thickened area at the proximal end of a condom, referred to as a.

13. The two layered condom as recited in claim 9, wherein said potential space is space between an inner condom and an outer condom, referred to as a.

14. The two layered condom as recited in claim 12, wherein said potential space is space between an inner condom and an outer condom, referred to as a.

15. A two layered condom for enhancing tactile stimulus transmission to the wearer while improving protection from unwanted pregnancy and the spread of sexually transmitted diseases comprising:
a condom barrier made of a smooth, flexible material impermeable to fluid, referred to as an inner condom, for providing an inner barrier through which tactile stimuli created by the deflection of a plurality of projections in an outer condom are transmitted to a wearer during use of a device;
a condom barrier made of a flexible material impermeable to fluid, referred to as an outer condom, for providing a second, outer barrier to afford an extra measure of protection from the spread of sexually transmitted diseases and unwanted pregnancy while containing projections on its inner surface, which deflect through the wall of an inner condom during use of a device to create tactile stimuli for the wearer;
a thickened area at the proximal end of a condom, referred to as a condom base, for retaining the position onto the base of the penis of a wearer during use of a device on either an inner condom or an outer condom individually, or alternatively can potentially serve as a common connection at the bases of both an inner condom and an outer condom, circumferentially molded to said outer condom, and circumferentially molded to said inner condom;
a space between an inner condom and an outer condom, referred to as a potential space, for containing a lubricant, adjacently positioned to said outer condom, and adjacently positioned to said inner condom;
a liquid or viscous gel, referred to as lubricant, for decreasing the coefficient of friction between an inner condom and an outer condom in such a way that it enhances deflection of projections through the wall of an inner condom and thereby enhances tactile stimulus transmission to the wearer while it also provides the potential for acting as a vehicle for a chemical or chemicals which function to neutralize sperm and the causative agents of sexually transmitted diseases, fluidly retained to said potential space; and
a convexity of any shape arising from the inner surface of an outer condom, referred to as a projection, for creating a tactile stimulus to the wearer of a device by deflecting the wall of an inner condom during use, integrally molded to said outer condom.
